(19) United States
(12) Patent Application Publication
(10) Pub. No.: US 2011/0009443 A1
(43) Pub. Date: Jan. 13, 2011

(54) Pyrazolospiroketone Acetyl-Coa Carboxylase Inhibitors

(76) Inventors: Kevin Daniel Freeman-Cook, Carlsbad, CA (US); Brian Matthew Samas, Groton, CT (US)

Correspondence Address: PFIZER INC.;PATENT DEPARTMENT, Bld 114 M/S 9114, EASTERN POINT ROAD, GROTON, CT, 06340, US

(21) Appl. No.: 12/934,396

(22) PCT Filed: May 18, 2009
(86) PCT No.: PCT/IB09/05659

§ 371 Date: Sep. 24, 2010

Related U.S. Application Data
(60) Provisional application No. 61/056,652, filed on May 28, 2008.
(60) Provisional application No. 61/058,689, filed on Jun. 4, 2008.
(60) Provisional application No. 61/171,112, filed on Apr. 21, 2009.

(51) Int. Cl. A61K 31/438; C07D 491/20; A61P 3/04; A61P 3/10; A61P 1/16; A61P 5/50

(52) U.S. Cl. 514/278; 546/17

(57) ABSTRACT

The invention provides compounds of Formula (1) or a pharmaceutically acceptable salt of said compound, wherein R¹, R², and R³ are as described herein; pharmaceutical compositions thereof; and the use thereof in treating mammals suffering from the condition of being overweight.

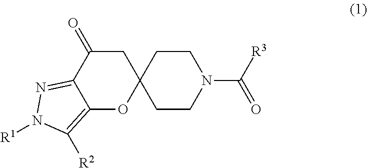

### FIELD OF THE INVENTION

This invention relates to a substituted pyrazolospiroketone compound that acts as an inhibitor of acetyl-CoA carboxylases and their use in treating diseases, conditions or disorders modulated by the inhibition of acetyl-CoA carboxylase enzyme(s).

### BACKGROUND OF THE INVENTION

Acetyl-CoA carboxylases (ACC) are a family of enzymes found in most species and are associated with fatty acid synthesis and metabolism through catalyzing the production of malonyl-CoA from acetyl-CoA. In mammals, two isoforms of the ACC enzyme have been identified. ACC1, which is expressed at high levels in lipogenic tissues, such as fat and the liver, controls the first committed step in the biosynthesis of long-chain fatty acids. If acetyl-CoA is not carboxylated to form malonyl-CoA, it is metabolized through the Krebs cycle. ACC2, which is a minor component of hepatic ACC but the predominant isoform in heart and skeletal muscle, catalyzes the production of malonyl-CoA at the cystolic surface of mitochondria, and regulates how much fatty acid is utilized in β-oxidation by inhibiting carnitine palmitoyl transferase. Thus, by increasing fatty acid utilization and by preventing increases in de novo fatty acid synthesis, chronic administration of an ACC inhibitor may also deplete liver and adipose tissue TG stores in obese subjects consuming a high or low-fat diet, leading to selective loss of body fat.

Studies conducted by Abu-Etheiga, et al., suggest that ACC2 plays an essential role in controlling fatty acid oxidation; therefore, ACC2 inhibition would provide a target for therapy against obesity and obesity-related diseases, such as type-2 diabetes. See, Abu-Etheiga, L., et al., “Acetyl-CoA carboxylase 2 mutant mice are protected against obesity and diabetes induced by high-fat/high-carbohydrate diets” PNAS, 100(18) 10207-10212 (2003). See also, Choi, C. S., et al., “Continuous fat oxidation in acetyl-CoA carboxylase 2 knockout mice increases total energy expenditure, reduces fat mass, and improves insulin sensitivity” PNAS, 104(42) 16480-16485 (2007). It is becoming increasingly clear that hepatic lipid accumulation causes hepatic insulin resistance and contributes to the pathogenesis of type 2 diabetes. Salvage, et al., demonstrated that ACC 1 and ACC2 are both involved in regulating fat oxidation in heptocytes while ACC1, the dominant isoform in rat liver, is the sole regulator of fatty acid synthesis. Furthermore, in their model, combined reduction of both isoforms is required to significantly lower hepatic malonyl-CoA levels, increase fat oxidation in the fed state, reduce lipid accumulation, and improve insultin action in vivo. Thus, showing that heptatic ACC1 and ACC2 inhibitors may be useful in the treatment of nonalcoholic fatty liver disease (NAFLD) and heptic insulin resistance. See, Savage, D. B., et al., “Reversal of diet-induced hepatic steatosis and hepatic insulin resistance by antisense oligonucleotide inhibitors of acetyl-CoA carboxylases 1 and 2” J Clin Invest doi: 10.1172/JCl27300. See also, Oh, W, et al., “Glucose and fat metabolism in adipose tissue of acetyl-CoA carboxylase 2 knowckout mice” PNAS, 102(5) 1384-1389 (2005).

Consequently, there is a need for medicaments containing ACC1 and ACC2 inhibitors to treat obesity and obesity-related diseases (such as, NAFLD and type-2 diabetes) by inhibiting fatty acid synthesis and by increasing fatty acid oxidation.

### SUMMARY OF THE INVENTION

The present invention relates to a compound having the structure of Formula (1) below.

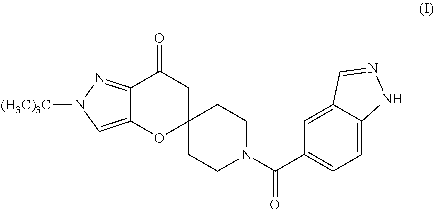

The compound of claim 1 may exist in a crystalline form having a powder X-ray diffraction pattern essentially the same as the pattern represented by FIG. 1 (having peaks at diffraction angle (2-theta) of 11.2±0.2, 15.4±0.2, 17.0±0.2, 18.3±0.2, 19.3±0.2 and 20.6±0.2). Referred to herein as polymorph Form A.

The compound of claim 1 may exist in a crystalline form having a powder X-ray diffraction pattern essentially the same as the pattern represented by FIG. 2 (having peaks at diffraction angle (2-theta) of 7.8±0.2, 11.2±0.2, 13.7±0.2, 15.9±0.2, 18.7±0.2 and 20.2±0.2). Referred to herein as polymorph Form B.

Another aspect of the present invention is a pharmaceutical composition that comprises (1) a compound of the present invention (including polymorphs Form A and B), and (2) a pharmaceutically acceptable excipient, diluent, or carrier. Preferably, the composition comprises a therapeutically effective amount of a compound of the present invention. The composition may also contain at least one additional pharmaceutical agent (described herein). Preferred agents include anti-obesity agents and/or anti-diabetic agents (described herein below).

In yet another aspect of the present invention is a method for treating a disease, condition, or disorder mediated by the inhibition of acetyl-CoA carboxylase enzyme(s) in a mammal that includes the step of administering to a mammal, preferably a human, in need of such treatment a therapeutically effective amount of a compound of the present invention, or a pharmaceutical composition thereof.

Diseases, disorders, or conditions mediated by inhibitors of acetyl-CoA carboxylases include Type II diabetes and diabetes-related diseases, such as nonalcoholic fatty liver disease (NAFLD), heptic insulin resistance, hyperglycemia, metabolic syndrome, impaired glucose tolerance, diabetic neuropathy, diabetic nephropathy, diabetic retinopathy, obesity, dyslididemia, hypertension, hyperinsulinemia, and insulin resistance syndrome. Preferred diseases, disorders, or conditions include Type II diabetes, nonalcoholic fatty liver disease (NAFLD), heptic insulin resistance, hyperglycemia, impaired glucose tolerance, obesity, and insulin resistance syndrome. More preferred are Type II diabetes, nonalcoholic fatty liver disease (NAFLD), heptic insulin resistance, hyperglycemia, and obesity. Most preferred is Type II diabetes.

A preferred embodiment is a method for treating or delaying the progression or onset of Type 2 diabetes and diabetes-related disorders in animals comprising the step of administering to an animal in need of such treatment a therapeutically effective amount of a compound of the present invention or a composition thereof.

Another preferred embodiment is a method for treating obesity and obesity-related disorders in animals comprising the step of administering to an animal in need of such treatment a therapeutically effective amount of a compound of the present invention or a composition thereof.

Yet another preferred embodiment is a method for treating nonalcoholic fatty liver disease (NAFLD) or heptic insulin resistance in animals comprising the step of administering to an animal in need of such treatment a therapeutically effective amount of a compound of the present invention or a composition thereof.

Compounds of the present invention may be administered in combination with other pharmaceutical agents (in particular, anti-obesity and anti-diabetic agents described herein below). The combination therapy may be administered as (a) a single pharmaceutical composition which comprises a compound of the present invention, at least one additional pharmaceutical agent described herein and a pharmaceutically acceptable excipient, diluent, or carrier; or (b) two separate pharmaceutical compositions comprising (i) a first composition comprising a compound of the present invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

### DEFINITIONS

The term “essentially the same” with reference to X-ray diffraction peak positions means that typical peak position and intensity variability are taken into account. For example, one skilled in the art will appreciate that the peak positions (2-theta) will show some inter-apparatus variability, typically as much as 0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The terms “modulated” or “modulating”, or “modulate(s)”, as used herein, unless otherwise indicated, refers to the inhibition of the Acetyl-CoA carboxylases (ACC) enzyme(s) with compounds of the present invention.

The terms “mediated” or “mediating” or “mediate(s)”, as used herein, unless otherwise indicated, refers to the treatment or prevention the particular disease, condition, or disorder, (ii) attenuation, amelioration, or elimination of one or more symptoms of the particular disease, condition, or disorder, or (iii) prevention or delay of the onset of one or more symptoms of the particular disease, condition, or disorder described herein, by inhibiting the Acetyl-CoA carboxylases (ACC) enzyme(s).

The term “compound of the present invention” (unless specifically identified otherwise) refers to a compound of Formula (I) as well as, all tautomers, conformational isomers, and isotopically labeled compounds. Hydrates and solvates of the compounds of the present invention are considered compositions of the present invention, wherein the compound is in association with water or solvent, respectively.

### DESCRIPTION OF THE FIGURES

FIG. 1 illustrates the powder X-ray diffraction pattern (pxrd) spectra for the Form A polymorph for the Compound of Formula (I).

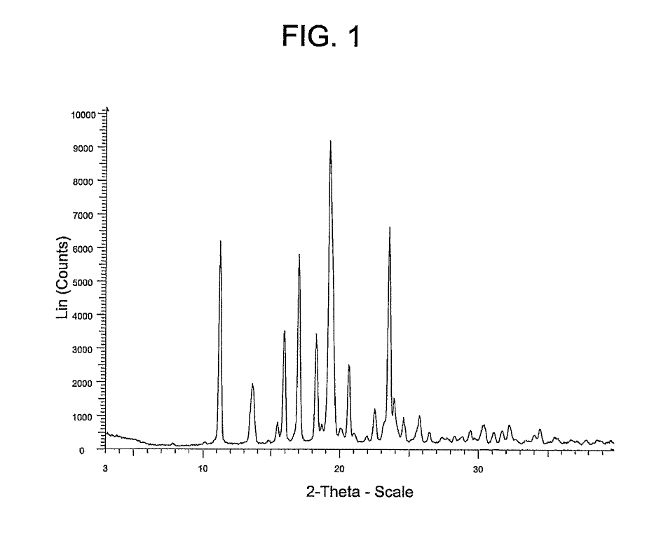

FIG. 2 illustrates the powder X-ray diffraction pattern (pxrd) spectra for the Form B polymorph for the Compound of Formula (I).

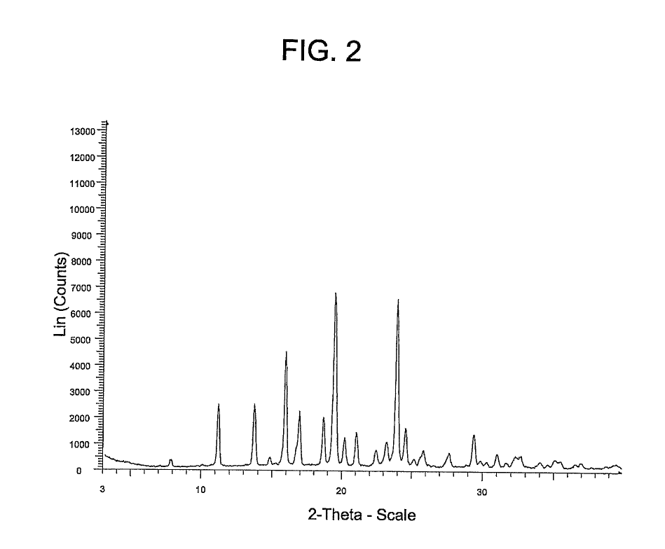

### DETAILED DESCRIPTION

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compound of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). Similarly, a “hydroxy-protecting group” refers to a substituent of a hydroxy group that blocks or protects the hydroxy functionality. Suitable hydroxyl-protecting groups (O-Pg) include for example, allyl, acetyl, silyl, benzyl, para-methoxybenzyl, trityl, and the like. The need for such protection is readily determined by one skilled in the art. For a general description of protecting groups and their use, see T. W. Greene, Protective Groups in Organic Synthesis, John Wiley & Sons, New York, 1991.

Scheme I outlines the general procedures one could use to provide the compound of the present invention having Formula (I).

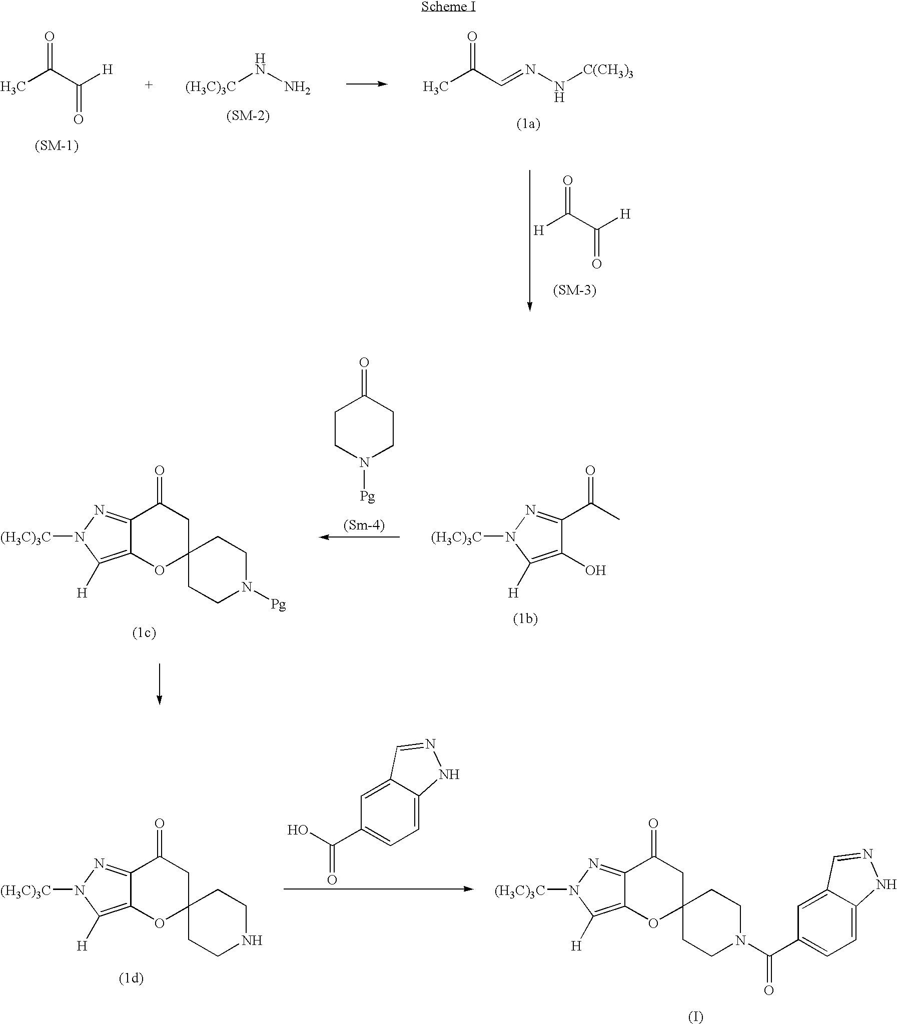

The intermediate hydrazone (1a) may be formed by treating methylglyoxal (SM-1) with 1-t-butylhydrazine (SM-2) in an acidic environment, such as acetic acid, at room temperature. Treatment of the hydrazone (1a) with oxalaldehyde (SM-3) in refluxing aqueous acetic acid provides the 1-(4-hydroxy-1H-pyrazole-3-yl)ethanone intermediate (1b). Alternatively, the 1H-pyrazole intermediate (1b) can also be formed directly by treating oxalaldehyde (SM-3) with 1-t-butylhydrazine oxalate in refluxing aqueous acetic acid. The amino-protected pyrazolospiroketone intermediate (1c) may be formed by adding an amino-protected 4-piperidone (preferably, a BOC protection group) to the 1-(4-hydroxy-1H-pyrazole-3-yl)ethanone intermediate (1b) in the presence of a an amine (preferably, pyrrolidine) at room temperature. The protecting group may then be removed to provide the pyrazolospiroketone intermediate (1d). The conditions used to remove the amino-protecting group will depend upon which protecting group was used. For example, a BOC protecting group can be removed by treatment with a strong acid (e.g., HCl). The final compound (I) may then be formed using a standard peptide coupling reaction with the 1H-indazole-5-carboxylic acid. For example, The pyrazolospiroketone intermediate (1d) and 1H-indazole-5-carboxylic acid may be coupled by forming an activated carboxylic acid ester, such as by contacting 1H-indazole-5-carboxylic acid with a peptide coupling reagent, such as O-(7-azabenzotriazol-1-yl)-N,N,N′,N′-tetramethyluronium hexafluorophosphate (HATU), in the presence or absence of an activating agent, such as hydroxybenzotriazole (HOBt) and in the presence of a suitable base, such as N,N-diisopropylethylamine (DIEA) or N-methylmorpholine (NMM), in a suitable solvent such as THF and/or DMF and then contacting the activated carboxylic acid ester with the pyrazolospiroketone intermediate (1d) to form a compound of Formula (1). Alternately, compounds of Formula (1) can be formed by first converting 1H-indazole-5-carboxylic acid to an acid chloride, such as by reacting with thionyl chloride, and then reacting the acid chloride with the pyrazolospiroketone intermediate (1d) to form a compound of Formula (1). Still another alternative entails treating 1H-indazole-5-carboxylic acid with 2-chloro-4,6-dimethoxytriazine in the presence of a suitable base, such as N-methylmorpholine in a suitable solvent such as THF and/or DMF. To the activated ester is added a solution of pyrazolospiroketone intermediate (1d) and base, such as N-methylmorpholine, in a suitable solvent, such as THF and/or DMF.

The compound of the present invention may exist in more than one crystal form. Polymorphs of the compounds of the present invention (including solvates and hydrates) form part of this invention and may be prepared by crystallization of a compound of the present invention under different conditions. For example, using different solvents or different solvent mixtures for recrystallization; crystallization at different temperatures; various modes of cooling, ranging from very fast to very slow cooling during crystallization. Polymorphs may also be obtained by heating or melting a compound of the present invention followed by gradual or fast cooling. The presence of polymorphs may be determined by solid probe nuclear magnetic resonance (NMR) spectroscopy, infrared (IR) spectroscopy, differential scanning calorimetry, powder X-ray diffraction or such other techniques.

This invention also includes isotopically-labeled compounds, which are identical to those described by Formula (1), but for the fact that one or more atoms are replaced by an atom having an atomic mass or mass number different from the atomic mass or mass number usually found in nature. Examples of isotopes that can be incorporated into the compound of Formula (I) include isotopes of hydrogen, carbon, nitrogen, oxygen, sulfur and fluorine, such as ²H, 3H, ¹³C, ¹⁴C, ¹⁵N, ¹⁸O, ¹⁷O, ³⁵S, ³⁶Cl, ¹²⁵I, ¹²⁹I, and ¹⁸F respectively. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of the present invention can generally be prepared by carrying out the procedures disclosed in the schemes and/or in the Examples below, by substituting a readily available isotopically labeled reagent for a non-isotopically labeled reagent.

The compounds of present invention may exist in different stable conformational forms which may be separable. Torsional asymmetry due to restricted rotation about an asymmetric single bond, for example because of steric hindrance or ring strain, may permit separation of different conformers. The compounds of the present invention further include each conformational isomer of the compound of Formula (1) and mixtures thereof.

Compounds of the present invention are useful for treating diseases, conditions and/or disorders modulated by the inhibition of the acetyl-CoA carboxylases enzyme(s) (in particular, ACC1 and ACC2); therefore, another embodiment of the present invention is a pharmaceutical composition comprising a therapeutically effective amount of a compound of the present invention and a pharmaceutically acceptable excipient, diluent or carrier. The compounds of the present invention (including the compositions and processes used therein) may also be used in the manufacture of a medicament for the therapeutic applications described herein.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The dissolution rate of poorly water-soluble compounds may be enhanced by the use of a spray-dried dispersion, such as those described by Takeuchi, H., et al. in “Enhancement of the dissolution rate of a poorly water-soluble drug (tolbutamide) by a spray-drying solvent deposition method and disintegrants” J. Pharm. Pharmacol., 39, 769-773 (1987); and EP0901786 B1 (U.S.2002/009494), incorporated herein by reference. The compound of the present invention is typically formulated into pharmaceutical dosage forms to provide an easily controllable dosage of the drug and to give the patient an elegant and easily handleable product.

The pharmaceutical compositions also include solvates and hydrates of the compound of the present invention. The term “solvate” refers to a molecular complex of a compound of the present invention with one or more solvent molecules. Such solvent molecules are those commonly used in the pharmaceutical art, which are known to be innocuous to the recipient, e.g., water, ethanol, ethylene glycol, and the like, The term “hydrate” refers to the complex where the solvent molecule is water. The solvates and/or hydrates preferably exist in crystalline form. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The present invention further provides a method of treating diseases, conditions and/or disorders modulated by the inhibition of the acetyl-CoA carboxylases enzyme(s) in an animal that includes administering to an animal in need of such treatment a therapeutically effective amount of a compound of the present invention or a pharmaceutical composition comprising an effective amount of a compound of the present invention and a pharmaceutically acceptable excipient, diluent, or carrier. The method is particularly useful for treating diseases, conditions and/or disorders that benefit from the inhibition of acetyl-CoA carboxylases enzyme(s).

One aspect of the present invention is the treatment of obesity, and obesity-related disorders (e.g., overweight, weight gain, or weight maintenance).

Obesity and overweight are generally defined by body mass index (BMI), which is correlated with total body fat and estimates the relative risk of disease. BMI is calculated by weight in kilograms divided by height in meters squared (kg/m²). Overweight is typically defined as a BMI of 25-29.9 kg/m², and obesity is typically defined as a BMI of 30 kg/m². See, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083 (1998).

Another aspect of the present invention is for the treatment or delaying the progression or onset of diabetes or diabetes-related disorders including Type 1 (insulin-dependent diabetes mellitus, also referred to as “IDDM”) and Type 2 (noninsulin-dependent diabetes mellitus, also referred to as “NIDDM”) diabetes, impaired glucose tolerance, insulin resistance, hyperglycemia, and diabetic complications (such as atherosclerosis, coronary heart disease, stroke, peripheral vascular disease, nephropathy, hypertension, neuropathy, and retinopathy).

In yet another aspect of the present invention is the treatment of obesity comorbidities, such as metabolic syndrome. Metabolic syndrome includes diseases, conditions or disorders such as dyslipidemia, hypertension, insulin resistance, diabetes (e.g., Type 2 diabetes), coronary artery disease and heart failure. For more detailed information on Metabolic Syndrome, see, e.g., Zimmet, P. Z., et al., “The Metabolic Syndrome: Perhaps an Etiologic Mystery but Far From a Myth—Where Does the International Diabetes Federation Stand?,” Diabetes & Endocrinology, 7(2), (2005); and Alberti, K. G., et al., “The Metabolic Syndrome—A New Worldwide Definition,” Lancet, 366, 1059-62 (2005). Preferably, administration of the compounds of the present invention provides a statistically significant (p<0.05) reduction in at least one cardiovascular disease risk factor, such as lowering of plasma leptin, C-reactive protein (CRP) and/or cholesterol, as compared to a vehicle control containing no drug. The administration of compounds of the present invention may also provide a statistically significant (p<0.05) reduction in glucose serum levels.

In yet another aspect of the invention is the treatment of nonalcoholic fatty liver disease (NAFLD) and heptic insulin resistance.

For a normal adult human having a body weight of about 100 kg, a dosage in the range of from about 0.001 mg to about 10 mg per kilogram body weight is typically sufficient, preferably from about 0.01 mg/kg to about 5.0 mg/kg, more preferably from about 0.01 mg/kg to about 1 mg/kg. However, some variability in the general dosage range may be required depending upon the age and weight of the subject being treated, the intended route of administration, the particular compound being administered and the like. The determination of dosage ranges and optimal dosages for a particular patient is well within the ability of one of ordinary skill in the art having the benefit of the instant disclosure. It is also noted that the compounds of the present invention can be used in sustained release, controlled release, and delayed release formulations, which forms are also well known to one of ordinary skill in the art.

The compounds of the present invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents (including appetite suppressants), anti-diabetic agents, anti-hyperglycemic agents, lipid lowering agents, and anti-hypertensive agents.

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY₃₋₃₆(including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Preferred anti-obesity agents for use in the combination aspects of the present invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or U.S. Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆(as used herein “PYY₃₋₃₆” includes analogs, such as peglated PYY₃₋₃₆ e.g., those described in U.S. Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Preferably, compounds of the present invention and combination therapies are administered in conjunction with exercise and a sensible diet.

Suitable anti-diabetic agents include a sodium-glucose co-transporter (SGLT) inhibitor, a phosphodiesterase (PDE)-10 inhibitor, a diacylglycerol acyltransferase (DGAT) 1 or 2 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., Byetta™, exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist and a glucokinase activator. Preferred anti-diabetic agents are metformin, a glucagon-like peptide 1 (GLP-1) agonist (e.g., Byetta™) and DPP-IV inhibitors (e.g., sitagliptin, vildagliptin, alogliptin and saxagliptin).

All of the above recited U.S. patents and publications are incorporated herein by reference.

The Examples set forth herein below are for illustrative purposes only. The compositions, methods, and various parameters reflected herein are intended only to exemplify various aspects and embodiments of the invention, and are not intended to limit the scope of the claimed invention in any way. Those of skill in the art would know how to optimize reagents, solvents and conditions based on the scale of the reaction and particular equipment used.

### EXAMPLES

The compounds and intermediates described below were generally named according to the IUPAC (International Union for Pure and Applied Chemistry) recommendations on Nomenclature of Organic Chemistry and the CAS Index rules. Unless noted otherwise, all reactants were obtained commercially. All of the references cited herein below are incorporated by reference.

Flash chromatography was performed according to the method described by Still et al., J. Org. Chem., 1978, 43, 2923.

All Biotage® purifications, discussed herein, were performed using either a 40M or 40S Biotage® column containing KP-SIL silica (40-63 μM, 60 Angstroms) (Bioatge AB; Uppsala, Sweden).

All Combiflash® purifications, discussed herein, were performed using a CombiFlash® Companion system (Teledyne Isco; Lincoln, Nebr.) utilizing packed RediSep® silica columns

Mass Spectra were recorded on a Waters (Waters Corp.; Milford, Mass.) Micromass Platform II spectrometer. Unless otherwise specified, mass spectra were recorded on a Waters (Milford, Mass.) Micromass Platform II spectrometer.

Proton NMR chemical shifts are given in parts per million downfield from tetramethylsilane and were recorded on a Varian Unity 400 or 500 MHz (megaHertz) spectrometer (Varian Inc.; Palo Alto, Calif.). NMR chemical shifts are given in parts per million downfield from tetramethylsilane (for proton) or fluorotrichloromethane (for fluorine).

### Key Intermediates and Starting Materials

1H-indazole-5-carboxylic acid is available from Tyger Scientific, Inc., Ewing, N.J.

### Preparation of Intermediate 2′-tert-Butyl2′H-spiro[piperidine-4,5′-pyrano[3,2-c]pyrazol]-7′(6′H)-one hydrochloride (I-1a)

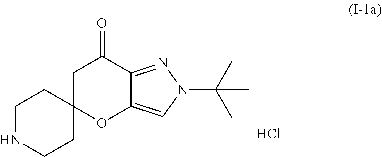

A solution of pyruvaldehyde (26.2 mL, 160 mmol) in H₂O (120 mL) was added to a solution of tert-butylhydrazine·HCl (20 g, 124 mmol) in H₂O (500 mL) over 20 minutes. This solution was stirred for 5 hours at room temperature. The reaction mixture was then extracted with ethyl acetate (5×). The combined organic layers were dried (Na₂SO₄) and concentrated under reduced pressure. The residue was then purified by flash chromatography (silica gel) eluting with a gradient of ethyl acetate/heptanes (10:90 to 40:60) to deliver 13.1 g (74%) of 2-oxopropanal tert-butylhydrazone as an amber oil.

A 40% aqueous solution of glyoxal (2.9 mL, 25.3 mmol) was added to 2-oxopropanal tert-butylhydrazone (1.20 g, 8.44 mmol) in water (14 mL). The mixture was then was heated at reflux for 5 hours. The reaction mixture was cooled to room temperature and extracted with EtOAc four times. The combined organic layers were dried (Na₂SO₄), filtered, and concentrated under reduced pressure. The residue was purified by flash chromatography (silica gel) and eluted with a gradient of heptanes/ethyl acetate (100:0 to 90:10) to yield 1.06 g (69%) of 1-(4-hydroxy-1-tert-butyl-1H-pyrazol-3-yl)ethanone as a colorless oil.

To a solution of 1-(4-hydroxy-1-tert-butyl-1H-pyrazol-3-yl)ethanone (6.63 g, 36.4 mmol) in MeOH (73 mL) was added pyrrolidine (3.6 mL, 43.7 mmol) and 1-(N-Boc)-4-piperidone (8.7 g, 43.7 mmol). The dark red solution was stirred at room temperature overnight. The solution was concentrated, the residue was dissolved in EtOAc and washed with 1 N NaOH and brine. The layers were separated and the organic layer was then set aside. The combined aqueous layers were extracted with ethyl acetate. The layers were separated, and the organic layer was washed with 1N NaOH and brine. All the organic layers were combined and then washed with 1 N HCl, and brine, dried (Na₂SO₄), filtered, and concentrated under reduced pressure to afford a red gum (11 g). The red gum was triturated and heated in 25% EtOAc/Hexanes (125 mL), the gum turned into yellow solid, but did not totally dissolve at reflux. The mixture was cooled to room temperature and filtered to yield an off-white solid (1.69 g) which was product. The filtrate was concentrated to around 5-10 mL (containing hexanes, EtOAc and Acetone) then an additional amount of 2% EtOAc/Hexanes (100 mL) was added whereupon a solid began to precipitate out. The mixture was stirred overnight. The solid was filtered to yield another 3.89 g of desired product as an off-white solid. In total, 5.58 g (42%) of tert-butyl 2′-tert-butyl-7′-oxo-6′,7′-dihydro-1H,2′H-spiro[piperidine-4,5′-pyrano[3,2-c]pyrazole]-1-carboxylate was isolated as an off-white solid.

To a solution of tert-butyl 2′-tert-butyl-7′-oxo-6′,7′-dihydro-1H,2′H-spiro[piperidine-4,5′-pyrano[3,2-c]pyrazole]-1-carboxylate (2.73 g, 7.5 mmol) in 1,4-dioxane (15 mL) at room temperature was added a solution HCl (4 M in 1,4-dioxane, 15 mL, 60 mmol). The mixture was stirred at room temperature for 3 hours. The reaction mixture was then concentrated to dryness. The resulting pink solid (2.6 g) was triturated with 2-methyltetrahydrofuran (20 mL) and a small amount of EtOH (1 mL). The solid was filtered, washed with 2- methyltetrahydrofuran (20 mL) and vacuum dried at 50° C. to yield 2.15 g (95%) of the title compound (I-1a)as a white solid.

### Example 1

### Preparation of 2′-tert-Butyl-1-(1H-indazol-5-ylcarbonyl)-2′H-spiro[piperidine-4,5′-pyrano[3,2-c]pyrazol]-7′(6′H)-one (I)

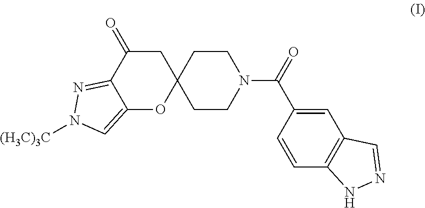

A mixture of 1 H-indazole-5-carboxylic acid (27 mg, 0.17 mmol), 2-chloro-4,6-dimethoxy-1,3,5-triazine (36 mg, 0.20 mmol) and N-methylmorpholine (NMM) (19 uL, 0.17 mmol) in N-dimethylformamide (1 mL) was stirred at room temperature for 35 minutes before addition of NMM (3 eq) followed by 2′-tert-butyl-2′H-spiro[piperidine-4,5′-pyrano[3,2-c]pyrazol]-7′(6′H)-one HCl (I-1a: 50 mg, 0.17 mmol). The mixture was stirred at room temperature overnight. The solvents were removed under reduced pressure, the residue dissolved in CH₂Cl₂ and washed with saturated aqueous NH₄Cl. The aqueous phase was back extracted with CH₂Cl₂ (2×). The combined organic extracts were washed with water and saturated aqueous NaCl before drying over MgSO₄. The material was filtered, concentrated and purified by preparative thin layer chromatography (95:5 CHCl₃/MeOH). The desired material was subsequently triturated with Et₂O, filtered and the solid was dried under vacuum at 50° C. to afford the desired product (21 mg, 31%).

¹H NMR (500 MHz, DMSO-d₆) δ ppm 13.26 (1 H, br. s.), 8.14 (1 H, s), 7.86 (1 H, s), 7.81 (1 H, s), 7.58 (1 H, d, J=8.54 Hz), 7.40 (1 H, br. s.), 3.18 (2 H, br. s.), 2.75 (2 H, s), 1.99 (2 H, s), 1.88 (2 H, br. s.), 1.74 (2 H, t), 1.51 (9 H, s).

### Example 2

Alternatively, Compound (I) may be prepared using the following procedure which produces a crystalline product (referred to herein as “Form A”).

To a 400 L reactor was charged: 2′-tert-butyl-2′H-spiro[piperidine-4,5′-pyrano[3,2-c]pyrazol]-7′(6′H)-one HCl (I-1a: 6.6 kg, 22.0 moles), 1H-indazole-5-carboxylic acid (3.26 kg, 20.1 moles), 1-(3-Dimethylaminopropyl)-3-ethylcarbodiimide Hydrochloride (4.85 kg, 25.3 moles), acetonitrile (124 L), and pyridine (13.9 L, 172 moles). Solution was stirred at ambient temperature for 16 hours, then diluted with ethyl acetate (250 L), and washed 2×'s 10-wt % aqueous citric acid (100 L). The organic layer was heated in order to distill to a 51 L solution volume, then added ethyl acetate (˜85 L) and distilled until an internal temperature of 76° C. was achieved (solution volume ˜55 L). The solution was then cooled to ambient temperature over 3 hours, the solids filtered through a Nutsche Filter, washed with ethyl acetate (17 L), and dried under vacuum at 50° C. for 12 hours. Compound (I) was isolated as a white crystalline solid (3.89 kg, 9.55 moles, 48%). MP: 265° C.

¹H NMR (400 MHz, DMSO-d₆) δ ppm 13.2 (1 H, s), 8.11 (1 H, s), 7.83 (1 H, s), 7.77 (1 H, s), 7.55 (1 H, d, J=8.0 Hz), 7.37 (1 H, dd, J=8.0, 1.2 Hz), 4.35-3.45 (2 H, m), 3.30-3.15 (2 H, m), 2.72 (2 H, s), 1.98-1.86 (2 H, m),1.73 (2 H, td, J=12.0, 4.0 Hz), 1.48 (9 H, s).

¹³C NMR (100 MHz, DMSO-d₆) δ ppm: 186.5, 170.2, 147.6, 140.6, 134.9, 134.2, 128.6, 125.9, 122.8, 120.5, 114.3, 110.7, 81.6, 61.0, 49.0, 34.0, 29.7.

The X-ray powder diffraction pattern for Form A polymorph of the Compound of Formula (I) was generated using a Siemens D5000 diffractometer with copper radiation. The instrument was equipped with a line focus X-ray tube. The tube voltage and amperage were set to 38 kV and 38 mA, respectively. The divergence and scattering slits were set at 1 mm, and the receiving slit was set at 0.6 mm. Diffracted Cu K_{α1} radiation (λ=1.54056 Å) was detected using a Sol-X energy dispersive X-ray detector. A theta two theta continuous scan at 2.4 ° 2θ/min (1 sec/0.04°2θ step) from 3.0 to 40°2θ was used. An alumina standard (NIST standard reference material 1976) was analyzed to check the instrument alignment. Data were collected and analyzed using BRUKER AXS DIFFRAC PLUS software Version 2.0. Samples were prepared for analysis by placing them in a quartz holder. It should be noted that Bruker Instruments purchased Siemens; thus, a Bruker D5000 instrument is essentially the same as a Siemens D5000. The table below summarizes the peaks having a 5× threshold over background observed for the

Form A crystal. The characterizing peaks (2-theta) for Form A are 11.2±0.2, 15.4±0.2, 17.0±0.2, 18.3±0.2, 19.3±0.2 and 20.6±0.2.

| Peak °2θ (+/−0.2) | Intensity % |
|---|---|
| 11.2 | 67.1 |
| 13.6 | 20.9 |
| 15.4 | 8.6 |
| 15.9 | 38.1 |
| 17.0 | 62.8 |
| 18.3 | 37.1 |
| 19.3 | 100 |
| 20.1 | 6.6 |
| 20.6 | 27.0 |
| 22.5 | 12.8 |
| 23.6 | 71.9 |
| 23.9 | 16.3 |
| 24.6 | 10.2 |
| 25.8 | 10.8 |
| 26.5 | 5.3 |
| 29.4 | 6.0 |
| 30.4 | 7.9 |
| 31.1 | 5.3 |
| 31.7 | 6.0 |
| 32.2 | 7.9 |
| 34.5 | 6.6 |

An eleven-fold increase in dissolution of Compound (I) was observed when Compound (I) was spray-dry dispersed (SDD) with hydroxypropylmethylcellulose acetate succinate (HPMCAS) in acetone (25% by wgt compound (I)). The compound and the SDD were compared and tested at 600 μg(active ingredient))/mL in a model fasted duodenal solution (0.5 wt % sodium taurocholate/1-palmitoyl-2-oleoyl-sn-glycero-3-phosphocholine in phosphate buffered saline, pH 6.5) by suspension in 0.5 wgt % Methocel™.

### Example 3

Example 3 provides a different polymorphic form of the Compound of Formula (I) (referred to herein as “Form B”).

Form A from Example 2 (20 mg) was added to a 4 mL vial containing a magnetic stir bar and 2 mL of acetone (2 mL). The solids were stirred for three weeks at 25° C. The solid was filtered on a PTFE filter; washed with 1 mL of MTBE. Approximately 10 mg of Form B was isolated as a white crystalline solid.

The X-ray powder diffraction pattern for Form B of the Compound of Formula (I) was generated using a Siemens D5000 diffractometer with copper radiation and the conditions described above in Example 2. The table below summarizes the peaks having a 5× threshold over background observed for the Form B polymorph. The characterizing peaks (2-theta) for Form B are 7.8±0.2, 11.2±0.2, 13.7±0.2, 15.9±0.2, 18.7±0.2 and 20.2±0.2.

| Peak °2θ (+/−0.2) | Intensity % |
|---|---|
| 7.8 | 5.2 |
| 11.2 | 37.5 |
| 13.7 | 36.8 |
| 14.8 | 7.1 |
| 15.9 | 67.0 |
| 16.9 | 32.9 |
| 18.7 | 29.3 |
| 19.4 | 100 |
| 20.2 | 18.3 |
| 21.0 | 22.0 |
| 22.5 | 11.3 |
| 23.2 | 16.0 |
| 23.9 | 96.8 |
| 24.6 | 23.8 |
| 25.2 | 6.4 |
| 25.9 | 11.4 |
| 27.6 | 10.1 |
| 29.4 | 20.5 |
| 31.0 | 9.6 |
| 32.3 | 8.4 |
| 32.6 | 8.3 |
| 34.1 | 5.3 |
| 35.1 | 6.6 |

### Pharmacological Data

### Biological Protocols

The utility of the compound of present invention, in the treatment of diseases is (such as are detailed herein) in animals, particularly mammals (e.g., humans) may be demonstrated by the activity thereof in conventional assays known to one of ordinary skill in the relevant art, including the in vitro and in vivo assays described below. Such assays also provide a means whereby the activities of the compound of the present invention can be compared with the activities of other known compounds.

### Direct Inhibition of the Activities of ACC1 and ACC2

The ACC inhibitory activity of the compound of the present invention was demonstrated by methods based on standard procedures. For example direct inhibition of ACC activity, for the compound of Formula (1) was determined using preparations of rat liver ACC and recombinant human ACC2.

[1] Preparation of rat liver ACC. Rat liver ACC was obtained from rat liver based upon standard procedures such as those described by Thampy and Wakil (J. Biol. Chem. 260: 6318-6323; 1985) using the following method.

Male CD rats weighing 150-200 g were fasted for 18-24 hours and then fed a high sucrose diet (AIN-76A rodent diet; Cat # D10001, Research Diets Inc., New Brunswick, N.J.), for 3 days at which time they were sacrificed by CO₂ asphyxiation. The livers were removed, rinsed in ice-cold phosphate-buffered saline (PBS), and homogenized in 5 volumes of homogenization buffer (50 mM potassium phosphate, pH 7.5, 10 mM EDTA, 10 mM 2-mercaptoethanol, 2 mM benzamidine, 0.2 mM phenylmethylsulfonylfluoride (PMSF), 5 mg/L each leupeptin, aprotinin, and antitrypsin) in a Waring® blender for 1 minute at 4° C. All subsequent operations were carried out at 4° C. The homogenate has made 3% with respect to polyethylene glycol (PEG) by the addition of 50% PEG solution and centrifuged at 20,000× g for 15 minutes. The resulting supernatant was adjusted to 5% PEG with the addition of 50% PEG solution and stirred for 5 minutes. The pellet (contains ACC activity) was collected by centrifugation at 20,000× g for 20 minutes, rinsed with ice-cold doubly distilled water to remove excess PEG and re-suspended in one-fourth the original homogenate volume with homogenization buffer. Ammonium sulfate (200 g/liter) was slowly added with stirring. After 45 minutes the enzyme is collected by centrifugation for 30 minutes at 20,000× g, re-suspended in 10 mL of 50 mM HEPES, pH 7.5, 0.1 mM DTT, 1.0 mM EDTA, and 10% glycerol and desalted on a Sephadex™ G-25 column (2.5 cm×50 cm) (Pharmacia, Piscataway N.J. now GE Healthcare) equilibrated with the same buffer. The desalted enzyme preparation was stored in aliquots at −70° C. Immediately prior to use, frozen rat liver ACC aliquots were thawed, diluted to 500 μg/mL in buffer containing 50 mM HEPES, pH 7.5, 10 mM MgCl₂, 10 mM tripotassium citrate, 2.0 mM dithiothreitol (DTT), and 0.75 mg/mL fatty acid-free bovine serum albumin (BSA) and pre-incubated at 37° C. for 30 minutes.

[2] Measurement of rat liver ACC inhibition. For measurement of ACC activity and assessment of ACC inhibition, test compounds were dissolved in dimethylsulfoxide (DMSO) and 1 μL aliquots were added to a clear bottom, 96-well plates (Perkin-Elmer PN#1450-514). Control wells contain 1 μL of DMSO alone or 1 μL of high inhibition compound. The enzyme obtained from rat liver as described above was activated in Enzyme buffer at 37° C. for 30 minutes prior to addition to compound plate. All wells receive 75 μL of activated enzyme (1.33×) in a buffer containing 50 mM HEPES, pH7.5, 7.5 mM MgCl₂ 7.5 mM tripotassium citrate, 2 mM DTT, 50 mg/mL BSA. The activated enzyme was pre-incubated with the compound for 10 minute prior to initiating the reaction through the addition of 25 μL of substrate solution containing 50 mM HEPES, pH 7.5, 7.5 mM MgCl₂ 7.5 mM tripotassium citrate, 2 mM DTT, 50 mg/mL BSA, 120 μM acetyl-CoA, 8.0 mM ATP, 38.4 mM KHCO₃, and 1.6 mM NaH[¹⁴C]O₃ (100 μCi/μL). The final substrate concentrations in the reaction were 30 μM Acetyl-CoA, 9.6 mM KHCO3, 0.4 mM NaH[¹⁴C]O₃, and 2 mM ATP. The reaction was terminated after 10 minutes by the addition of 25 μL 3N HCl and the plates were dried at 50° C. for a minimum 20 hours. 30 μL of water was added to the dried plate and mixed for 5 minutes. 95 μL of Optiphase Supermix liquid scintillation fluid (Perkin Elmer, Waltham, Mass.) was added and the plates are mixed for 20 minutes. Incorporation of ¹⁴C into MCoA was measured using a Wallac Trilux 1450 Microbeta LSC luminescence counter.

[3] Measurement of human ACC2 inhibition. Human ACC2 inhibition was measured using purified recombinant human ACC2 (hrACC2). Briefly, a full length Cytomax clone of ACC2 was purchased from Cambridge Bioscience Limited and was sequenced and subcloned into PCDNA5 FRT TO-TOPO (Invitrogen, Carlsbad, Calif.). The ACC2 was expressed in CHO cells by tetracycline induction and harvested in 5 liters of DMEM/F12 with glutamine, biotin, hygromycin and blasticidin with 1 μg/mL tetracycline (Invitrogen, Carlsbad, Calif.). The conditioned medium containing ACC2 was then applied to a Softlink Soft Release Avidin column (Promega, Madison, Wis.) and eluted with 5 mM biotin. 4 mgs of ACC2 were eluted at a concentration of 0.05 mg/mL (determined by A280) with an estimated purity of 95% (determined by A280). The purified ACC2 was dialyzed in 50 mM Tris, 200 mM NaCl, 4 mM DTT, 2 mM EDTA, and 5% glycerol. The pooled protein was frozen and stored at −80° C., with no loss of activity upon thawing. For measurement of ACC2 activity and assessment of ACC2 inhibition, test compounds were dissolved in DMSO and added to the rhACC2 enzyme as a 5× stock with a final DMSO concentration of 1%. rhACC2 was assayed in a Costar #3767 (Costar, Canbridge, Mass.) 384-well plate using the Transcreener ADP detection FP assay kit (Bellbrook Labs, Madison, Wis.) using the manufactures' conditions for a 50 μM ATP reaction. The final conditions for the assay were 50 mM HEPES, pH 7.5, 5 mM MgCl₂, 5 mM tripotassium citrate, 2 mM DTT, 0.5 mg/mL BSA, 30 μM acetyl-CoA, 50 μM ATP, and 8 mM KHCO₃. Typically, a 10 μL reaction was run for 1 hour at room temperature, and 10 μl of Transcreener stop and detect buffer was added and incubated for an additional 1 hour. The data was acquired on a Envision Fluorescence reader (Perkinelmer) using a 620 excitation Cy5 FP general dual mirror, 620 excitation Cy5 FP filter, 688 emission (S) and a 688 (P) emission filter.

The results using the rat liver ACC radio enzymatic and recombinant hACC2 transcreener assays described above are summarized in the table below for the Compound of Formula (I).

| | | Rat liver | Rat liver | | |
|---|---|---|---|---|---|
| | | ACC | ACC | rhACC2 | rhACC2 |
| Ex. | Compound Name | IC₅₀ (nM) | n* | IC₅₀ (nM) | n* |
| 1 | 2′-tert-Butyl-1-(1H- | 17.2 | 8 | 6.7 | 7 |
| | indazol-5-ylcarbonyl)- | | | | |
| | 2′H-spiro[piperidine- | | | | |
| | 4,5′-pyrano[3,2- | | | | |
| | c]pyrazol]-7′(6′H)-one | | | | |

*n is the number of replications.

### Acute in vivo Assessment of ACC Inhibition in Experimental Animals

The ACC inhibitory activity of the compound of the present invention can be confirmed in vivo by evaluation of their ability to reduce malonyl-CoA levels in liver and muscle tissue from treated animals.

Measurement of malonyl-CoA production inhibition in experimental animals. In this method, male Sprague-Dawley Rats, maintained on standard chow and water ad libitum (225-275g), were randomized prior to the study. Animals were either fed, or fasted for 18 hours prior to the beginning of the experiment. Two hours into the light cycle the animals were orally dosed with a volume of 5 mL/kg, (0.5% methyl cellulose; vehicle) or with the appropriate compound (prepared in vehicle). Fed vehicle controls were included to determine baseline tissue malonyl-CoA levels while fasted animals were included to determine the effect fasting had on malonyl-CoA levels. One hour after compound administration the animals were asphyxiated with CO₂ and the tissues were removed. Specifically, blood was collected by cardiac puncture and placed into BD Microtainer tubes containing EDTA (BD Biosciences, N.J.), mixed, and placed on ice. Plasma was used to determine drug exposure. Liver and quadriceps were removed, immediately freeze-clamped, wrapped in foil and stored in liquid nitrogen.

Tissues were pulverized under liquid N₂ to ensure uniformity in sampling. Malonyl-CoA was extracted from the tissue (150-200 mg) with 5 volumes 10% tricarboxylic acid in Lysing Matrix A (MP Biomedicals, PN 6910) in a FastPrep FP120 (Thermo Scientific, speed=5.5; for 45 seconds). The supernatant containing malonyl-CoA was removed from the cell debris after centrifugation at 15000× g for 30 minutes (Eppendorf Centrifuge 5402). Samples were stably frozen at −80° C. until analysis is completed.

Analysis of malonyl CoA levels in liver and muscle tissue can be evaluated using the following methodology.

The method utilizes the following materials: Malonyl-CoA tetralithium salt and malonyl-¹³C₃-CoA trilithium salt which were purchased from Isotec (Miamisburg, Ohio, USA), sodium perchlorate (Sigma, cat no. 410241), trichloroacetic acid (ACROS, cat no. 42145), phosphoric acid (J. T. Baker, cat no. 0260-01), ammonium formate (Fluka, cat no. 17843), methanol (HPLC grade, J. T. Baker, cat no. 9093-33), and water (HPLC grade, J. T. Baker, 4218-03) were used to make the necessary mobile phases. Strata-X on-line solid phase extraction columns, 25 μm, 20 mm×2.0 mm I.D (cat no. 00M-S033-B0-CB) were obtained from Phenomenex (Torrance, Calif., USA). SunFire C18 reversed-phase columns, 3.5 μm, 100 mm×3.0 mm I.D. (cat no.186002543) were purchased from Waters Corporation (Milford, Mass., USA).

This method may be performed utilizing the following equipment. Two-dimensional chromatography using an Agilent 1100 binary pump, an Agilent 1100 quaternary pump and two Valco Cheminert 6-port two position valves. Samples were introduced via a LEAP HTC PAL auto sampler with Peltier cooled stack maintained at 10° C. and a 20 μL sampling loop. The needle wash solutions for the autosampler are 10% trichloroacetic acid in water (w/v) for Wash 1 and 90:10 methanol:water for Wash 2. The analytical column (Sunfire) was maintained at 35° C. using a MicroTech Scientific Micro-LC Column Oven. The eluant was analyzed on an ABI Sciex API3000 triple quadrupole mass spectrometer with Turbo Ion Spray.

Two-dimensional chromatography was performed in parallel using distinct gradient elution conditions for on-line solid phase extraction and reversed-phase chromatography. The general design of the method was such that the first dimension was utilized for sample clean-up and capture of the analyte of interest followed by a brief coupling of both dimensions for elution from the first dimension onto the second dimension. The dimensions were subsequently uncoupled allowing for gradient elution of the analyte from the second dimension for quantification while simultaneously preparing the first dimension for the next sample in the sequence. When both dimensions were briefly coupled together, the flow of the mobile phase in the first dimension was reversed for analyte elution on to the second dimension, allowing for optimal peak width, peak shape, and elution time.

The first dimension of the HPLC system utilized the Phenomenex strata-X on-line solid phase extraction column and the mobile phase consisted of 100 mM sodium perchlorate/0.1% (v/v) phosphoric acid for solvent A and methanol for solvent B.

The second dimension of the HPLC system utilized the Waters SunFire C18 reversed-phase column and the mobile phase consisted of 100 mM ammonium formate for solvent A and methanol for solvent B. The initial condition of the gradient was maintained for 2 minutes and during this time the analyte was transferred to the analytical column. It was important that the initial condition was at a sufficient strength to elute the analyte from the on-line SPE column while retaining it on the analytical. Afterwards, the gradient rose linearly to 74.5% A in 4.5 minutes before a wash and re-equilibration step.

Mass spectrometry when coupled with HPLC can be a highly selective and sensitive method for quantitatively measuring analytes in complex matrices but is still subject to interferences and suppression. By coupling a two dimensional HPLC to the mass spectrometer, these interferences were significantly reduced. Additionally, by utilizing the Multiple Reaction Monitoring (MRM) feature of the triple quadrupole mass spectrometer, the signal-to-noise ratio was significantly improved.

For this assay, the mass spectrometer was operated in positive ion mode with a TurbolonSpray voltage of 2250V. The nebulizing gas was heated to 450° C. The Declustering Potential (DP), Focusing Potential (FP), and Collision Energy (CE) were set to 60, 340, and 42 V, respectively. Quadrupole 1 (Q1) resolution was set to unit resolution with Quadrupole 3 (Q3) set to low. The CAD gas was set to 8. The MRM transitions monitored were for malonyl CoA: 854.1→347.0 m/z (L. Gao et al. (2007) J. Chromatogr. B 853,303-313); and for malonyl-¹³C₃-CoA: 857.1→350.0 m/z with dwell times of 200 ms. The eluant was diverted to the mass spectrometer near the expected elution time for the analyte, otherwise it was diverted to waste to help preserve the source and improve robustness of the instrumentation. The resulting chromatograms were integrated using Analyst software (Applied Biosystems). Tissue concentrations for malonyl CoA were calculated from a standard curve prepared in a 10% solution of trichloroacetic acid in water.

Samples comprising the standard curve for the quantification of malonyl-CoA in tissue extracts were prepared in 10% (w/v) trichloroacetic acid (TCA) and ranged from 0.01 to 1 pmol/μL. Malonyl-¹³C₃-CoA (final concentration of 0.4 pmol/μL) was added to each standard curve component and sample as an internal standard.

Six intra-assay quality controls were prepared; three from a pooled extract prepared from fasted animals and three from a pool made from fed animals. These were run as independent samples spiked with 0, 0.1 or 0.3 pmol/μL ¹²C-malonyl-CoA as well as malonyl-¹³C₃-CoA (0.4 pmol/μL). Each intra-assay quality control contained 85% of aqueous tissue extract with the remaining portion contributed by internal standard (0.4 pmol/μL) and ¹²C-malonyl-CoA. Inter assay controls were included in each run; they consist of one fasted and one fed pooled sample of quadriceps and/or one fasted and one fed pooled sample of liver. All such controls are spiked with malonyl-¹³C₃-CoA (0.4 pmol/μL).

The compound of Formula (I) was used in the in vivo test described above to determine their effect upon malonyl CoA levels in liver and muscle tissue. The results are provided in the following table.

### Percent Decrease in Tissue Malonyl-CoA Levels

| | | Muscle Malonyl- | |
|---|---|---|---|
| | | CoA | Liver Malonyl- |
| Compound | Dose | (quadriceps)^{^{(a)}} | CoA^{^{(a)}} |
| Example 1 | 1 mg/kg | 2.34 (5.44) | 34.97 (2.59) |
| | 3 mg/kg | 24.42 (6.83) | 53.97 (1.19) |
| | 10 mg/kg | 49.00 (2.40) | 70.74 (3.49) |
| | 30 mg/kg | 57.06 (2.04) | 63.60 (3.57) |

^{^{(a)}}percent decrease in tissue malonyl-CoA relative to chow-fed vehicle control group (% decrease +/− SEM)

1. The compound of Formula (I)

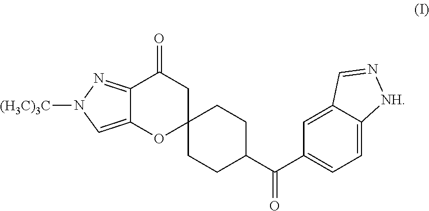

2. The compound of claim 1 wherein said compound is a crystalline form having a powder X-ray diffraction pattern comprising peaks at diffraction angle (2theta) of 11.2±0.2, 15.4±0.2, 17.0±0.2, 18.3±0.2, 19.3±0.2 and 20.6±0.2.

3. The compound of claim 1 wherein said compound is a crystalline form having a powder X-ray diffraction pattern comprising peaks at diffraction angle (2-theta) of 7.8±0.2, 11.2±0.2, 13.7±0.2, 15.9±0.2, 18.7±0.2 and 20.2±0.2.

4. A pharmaceutical composition comprising (i) a compound according to claim 1; and (ii) a pharmaceutically acceptable excipient, diluent, or carrier.

5. The composition of claim 4 wherein said compound is present in a therapeutically effective amount.

6. The composition of claims 5 further comprising at least one additional pharmaceutical agent selected from the group consisting of an anti-obesity agent and an anti-diabetic agent.

7. The composition of claim 6 wherein said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine.

8. The composition of claim 6 wherein said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

9. A method for treating obesity and obesity-related disorders in animals comprising the step of administering to an animal in need of such treatment a therapeutically effective amount of a compound of claim 1.

10. A method for treating or delaying the progression or onset of Type 2 diabetes and diabetes-related disorders in animals comprising the step of administering to an animal in need of such treatment a therapeutically effective amount of a compound of claim 1.

11. A method for treating nonalcoholic fatty liver disease (NAFLD) or heptic insulin resistance in animals comprising the step of administering to an animal in need of such treatment a therapeutically effective amount of a compound of claim 1.

12. A method for treating obesity and obesity-related disorders in animals comprising the step of administering to an animal in need of such treatment a pharmaceutical composition of claim 5.

13. A method for treating or delaying the progression or onset of Type 2 diabetes and diabetes-related disorders in animals comprising the step of administering to an animal in need of such treatment a pharmaceutical composition of claim 5.

14. A method for treating nonalcoholic fatty liver disease (NAFLD) or heptic insulin resistance in animals comprising the step of administering to an animal in need of such treatment a pharmaceutical composition of claim 5.

15-19. (canceled)
